(19) United States
(12) Patent Application Publication
(10) Pub. No.: US 2022/0111375 A1
(43) Pub. Date: Apr. 14, 2022

(54) ANTIFOULING CATALYST SYSTEMS FOR SELECTIVE ETHYLENE TRIMERIZATION

(71) Applicant: Saudi Arabian Oil Company, Dhahran (SA)

(72) Inventors: Sohel Shaikh, Dhahran (SA); Motaz Khawaji, Thuwal (SA); Zhonglin Zhang, Dhahran (SA); Wei Xu, Thuwal (SA)

(73) Assignee: Saudi Arabian Oil Company, Dhahran (SA)

(21) Appl. No.: 17/066,830

(22) Filed: Oct. 9, 2020

(51) Int. Cl. B01J 31/34; B01J 23/26; B01J 21/02; B01J 31/14; B01J 31/02; C07C 2/32

(52) U.S. Cl. CPC B01J 31/34; B01J 23/26; B01J 21/02; B01J 31/143; B01J 31/0238; B01J 31/0231; B01J 2231/20; C07C 2531/14; C07C 2531/22; C07C 2531/34; B01J 2531/31; B01J 2531/62; C07C 2/32

(57) ABSTRACT

According to one embodiment, a catalyst system that reduces polymeric fouling may include at least one chromium compound, at least one aluminum compound, and at least one antifouling agent or a derivative thereof. The antifouling agent may have a structure including a central aluminum molecule bound to an R1 group, bound to an R2 group, and bound to an R3 group. One or more of the chemical groups R1, R2, and R3 may be antifouling groups having the structure —O((CH₂)ₙO)ₘR4, a phosphonium or phosphonium salt, a sulfonate or sulfonate salt, a sulfonium or sulfonium salt, an ester, an anhydride, a polyether, or a long-chained amine-capped compound, where n is an integer from 1 to 20, m is an integer from 1 to 100, and R4 is a hydrocarbyl group. The chemical groups R1, R2, or R3 that do not include an antifouling group, if any, may be hydrocarbyl groups.

### BACKGROUND

### Field

Embodiments of the present disclosure generally relate to catalyst systems used in ethylene oligomerization and, more specifically, relate to antifouling catalyst systems used in ethylene oligomerization for generation of 1-hexene which reduce undesired polymerization.

### Technical Background

1-hexene is an important petrochemical, especially for the production of polyolefins. The reaction of ethylene and other alpha-olefins, such as 1-hexene, forms various grades of linear low density polyethylene (LLDPE), a useful commercial polymer. A source of 1-hexene is the hexene fraction from the effluent of a hydrocarbon cracker, such as a steam cracker or fluidized catalytic cracker. However, the process for recovering 1-hexene from such an effluent requires several difficult process steps that may make the process undesirable.

Several commercial processes selectively oligomerize ethylene into alpha olefins such as 1-butene and 1-hexene. A commercially successful dimerization process is the Alphabutol™ Process, developed by the Institute Francais du Petrole (IFP), described in A. Forestiere, et al., “Oligomerization of Monoolefins by Homogenous Catalysts”, Oil & Science and Technology—Review de l'Institute Francais du Petrole, pages 663-664 (Volume 64, Number 6, November 2009). This process uses a bubble-point reactor that contains 1-butene as a process fluid to oligomerize ethylene selectively into 1-butene. Similar processes are applicable to trimerization.

There is a known problem with oligomerization systems: polymer formation. Long residence times and poor heat removal from the highly exothermic reactions lead to the formation of polyethylene-based residues. A side effect of chronic fouling is increasingly frequent process shutdowns and higher maintenance costs for removing adhered polymer residues. Polymer residues may build layer upon layer and eventually close off openings and ports in locations with fluid flow. Additionally, a polymer coating along the wall of a reactor may act as an insulator, which may negatively affect heat transfer to the reactor system. Polymer can also collect debris that can be catalytically active or that can poison the reaction process.

An especially troublesome issue is the formation of “hot spots”. A hot spot is an area where external cooling is ineffective and catalyst activity is high. It represents a loss of process control. A hot spot can be an area of collected polymer that includes catalytically active material that fosters side-reactions, including polymerization. If left unchecked, the hot spot can eventually lead to a process shutdown due to the loss of cooling capacity, a runaway polymerization reaction, or both.

### SUMMARY

There is a continual need for effective methods to prevent polymeric fouling on reactor system walls and tubes while maintaining the desired oligomerization rate and selectivity to form reaction product.

According to one embodiment, a catalyst system that reduces polymeric fouling in the production of 1-hexene may comprise at least one chromium compound, at least one aluminum compound, and at least one antifouling agent or a derivative thereof. The antifouling agent may comprise a structure comprising a central aluminum molecule bound to an R1 group, bound to an R2 group, and bound to an R3 group. One or more of the chemical groups R1, R2, and R3 may be antifouling groups comprising the structure —O((CH₂)ₙO)ₘR4, a phosphonium or phosphonium salt, a sulfonate or sulfonate salt, a sulfonium or sulfonium salt, an ester, an anhydride, a polyether, or a long-chained amine-capped compound, where n is an integer from 1 to 20, m is an integer from 1 to 100, and R4 is a hydrocarbyl group. The chemical groups R1, R2, or R3 that do not comprise the antifouling group, if any, may be hydrocarbyl groups.

According to another embodiment, 1-hexene may be produced by a process comprising contacting ethylene with a catalyst system to oligomerize the ethylene to form 1-hexene. The catalyst system may comprise at least one chromium compound, at least one aluminum compound, and at least one antifouling agent or a derivative thereof. The antifouling agent may comprise a structure comprising a central aluminum molecule bound to an R1 group, bound to an R2 group, and bound to an R3 group. One or more of the chemical groups R1, R2, and R3 may be antifouling groups comprising the structure —O((CH₂)ₙO)ₘR4, a phosphonium or phosphonium salt, a sulfonate or sulfonate salt, a sulfonium or sulfonium salt, an ester, an anhydride, a polyether, or a long-chained amine-capped compound, where n is an integer from 1 to 20, m is an integer from 1 to 100, and R4 is a hydrocarbyl group. The chemical groups R1, R2, or R3 that do not comprise the antifouling group, if any, may be hydrocarbyl groups.

Additional features and advantages of the embodiments described herein will be set forth in the detailed description which follows, and in part will be readily apparent to those skilled in the art from that description or recognized by practicing the embodiments described, including the detailed description which subsequently follows, and the claims.

### BRIEF DESCRIPTION OF THE DRAWINGS

The following detailed description of specific embodiments of the present disclosure can be best understood when read in conjunction with the following drawings in which:

FIG. 1 is a graphical depiction of the effect of AFA:Cr ratio on polymer formation;

FIG. 2 is a graphical depiction of the effect of AFA:Cr ratio on trimerization activity; and

FIG. 3 is a graphical depiction of the effect of AFA:Cr ratio on 1-hexene selectivity.

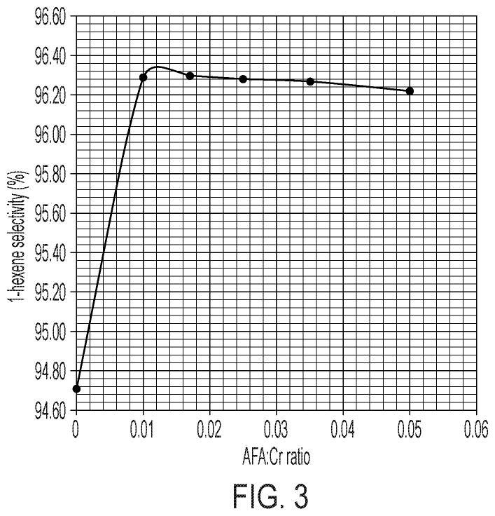

### DETAILED DESCRIPTION

One or more embodiments of the present disclosure are directed to catalyst systems which may be utilized in promoting ethylene oligomerization, such as the trimerization of ethylene to form 1-hexene, while reducing reactor fouling caused by undesired polymerization. These catalyst systems are sometimes referred to in this disclosure as “antifouling ethylene oligomerization catalyst systems” or “antifouling catalyst systems”. The antifouling catalyst systems described may comprise at least one chromium compound, at least one aluminum compound, and at least one antifouling agent or dimer thereof. The antifouling catalyst systems may further comprise one or more halogen containing compounds. Further, the antifouling catalyst systems may further comprise one or more nitrogen containing compounds. The antifouling catalyst systems may be used to selectively oligomerize ethylene to produce 1-hexene, while reducing undesirable polymerization, sometimes referred to in this disclosure as “fouling”. For example, reactor fouling may occur due to the formation of solid polyethylene-based residues which may reduce fluid flow and fully block or at least partially block fluids in a reactor system from flowing at a desired rate. It should be understood that the “antifouling ethylene oligomerization catalyst systems” or “antifouling catalyst systems” described may not completely eliminate fouling during a reaction. However, these catalyst systems reduce fouling as compared with catalyst systems which do not include an antifouling agent as described in the present disclosure. Also, it should be understood that while the catalyst systems of the present disclosure may be useful in ethylene oligomerization reactions, such as ethylene trimerization to form 1-hexene, they may also be useful for the catalysis of other chemical reaction, and the antifouling catalyst systems described in this disclosure should not be considered limited in their use to the trimerization of ethylene to 1-hexene.

As described previously in this disclosure, embodiments of the described antifouling catalyst systems may comprise one or more chromium compounds. While several chromium compounds may be included in the antifouling catalyst system, in some embodiments a single chromium compound may be included in the antifouling catalyst system. In one or more embodiments, the chromium compound may be one or more of alkoxy salts of chromium, carboxyl salts of chromium. B-diketonate salts of chromium, salts of chromium and an anion derived from B-keto-esters, and chromium halides. Suitable chromium compounds may include chromium (IV)-tert-butoxide, chromium (III)-acetylacetonate, chromium (III)-trifluoro acetylacetonate, chromium (III)-hexafluoro acetylacetonate, chromium (III) (2,2,6,6-tetramethyl-3,5-heptane-dionate), Cr(PhCOCHCOPh) wherein Ph represents a phenyl group, chromium (II) acetate, chromium (III) acetate, chromium (III)-2-ethyl hexanoate, chromium (III) benzoate, chromium (III) naphthenate, Cr(CH—COCHCOOCH), chromium (II) chloride, chromium(III) chloride, chromium (II) bromide, chromium (III)bromide, chromium(II) iodide, chromium(III) iodide, chromium (II) fluoride, and chromium (III) fluoride. In one or more embodiments, the chromium compound of the antifouling catalyst system consists of a complex of chromium and 2-ethyl hexanoate [Cr—[CH₃(CH₂)₃CH(C₂H₅)CO₂]₃] as graphical represented by Chemical Structure #1.

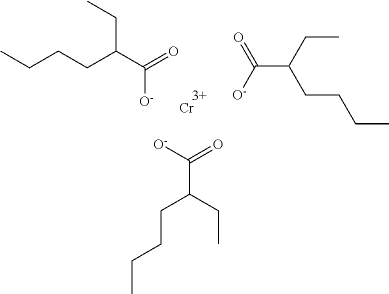

### Chemical Structure #1—Complex of Chromium and 2-Ethyl Hexanoate

As also described previously in this disclosure, embodiments of the described antifouling catalyst systems may comprise one or more aluminum compounds. While several aluminum compounds may be included in the antifouling catalyst system, in some embodiments a single aluminum compound may be included. In one or more embodiments, one or more aluminum alkyl compounds may be included in the antifouling catalyst system. Aluminum alkyl compounds may have a structure of AlR′₃ or AlR′₂H, where R′ is a straight chain or branched alkane comprising from 1 to 20 carbons, or an aluminoxane structure (that is, a partial hydrolysate of trialkylaluminum compounds). For example, and not by way of limitation, suitable aluminum alkyl compounds may include triethylaluminum, tripropylaluminum, tri-iso-butylaluminum, and trihexylaluminum. In one or more embodiments, the aluminum compound of the antifouling catalyst system consists of triethylaluminum.

The antifouling catalyst systems may comprise one or more antifouling agents or derivatives thereof. As used herein, a derivative refers to a derivative structure of an antifouling agent, such as a dimer, trimer, oligomer, polymer, isomer, hydrolysate of an antifouling agent described in this disclosure. In one or more embodiments, an antifouling agent may comprise a central aluminum molecule bonded to all three of a first chemical group R1, a second chemical group R2, and a third chemical group R3. The first chemical group R1, the second chemical group R2, and the third chemical group R3 represent antifouling groups. Antifouling groups contemplated include phosphoniums, sulfonates, sulfoniums, esters, anhydrides, polyethers, long-chained amine-capped compounds, and antifouling groups comprising the structure —O((CH₂)ₙO)ₘR4 as detailed infra. It should be understood that as used in this disclosure, antifouling agents which are named for a particular chemical moiety (for example, a “sulfonate antifouling agent” or a “phosphonium antifouling agent”) comprise at least one of that particular chemical moiety but may include additional chemical moieties. For example, a “sulfonate antifouling agent” is an antifouling agent which includes a sulfonate moiety and a “phosphonium antifouling agent” is an antifouling agent which includes a phosphonium moiety. Chemical Structure #2 depicts a generalized chemical structure of an antifouling agent.

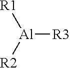

### Chemical Structure #2—Generalized Antifouling Agent

In one or more embodiments, one or more of R1, R2, and R3 are antifouling groups comprising the structure —O((CH₂)ₙO)ₘR4, where n is an integer from 1 to 20 (for example, 1 to 15, 1 to 10, 1 to 5, 1 to 4, 1 to 3, 1 to 2, 2 to 20, 3 to 20, 4 to 20, 5 to 20, 10 to 20, or 15 to 20), m is an integer from 1 to 100 (for example, 1 to 10, 1 to 20, 1 to 30, 1 to 40, 1 to 50, 1 to 75, 5 to 100, 10 to 100, 25 to 100, 50 to 100, or 75 to 100), and R4 is a hydrocarbyl group. The structure of the antifouling group, —O((CH₂)ₙO)ₘR4, is depicted in Chemical Structure #3. The central aluminum atom is bonded to a terminal oxygen of the antifouling group opposite of the R4 hydrocarbyl group. As used throughout this disclosure, a hydrocarbyl group refers to a chemical group that consists of hydrogen and carbon atoms. For example, a hydrocarbyl group may be branched or unbranched, and may comprise one or more alkane moieties, one or more alkene moieties, one or more alkyne moieties, or combinations thereof. Hydrocarbyl groups may include cyclic or aromatic moieties. In one or more embodiments, R4 may be a hydrocarbyl group having from 1 to 100 carbon atoms, such as from 5 to 50 carbon atoms, or from 12 to 28 carbon atoms.

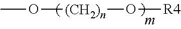

### Chemical Structure #3—Antifouling Group

In one or more embodiments, one or more of R1, R2, and R3 are antifouling groups comprising one or more phosphonium compounds. As used in this disclosure, phosphonium antifouling agents include any compound comprising the phosphonium structure depicted in Chemical Structure #4, where R₆, R₇, R₈, and R₉ represents chemical groups which may contain other moieties. R₆, R₇, R₈, and R₉ may represent branched or unbranched alkanes, alkenes, aryls, or halides and the R groups may be identical or different from one another.

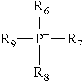

### Chemical Structure #4—Generalized Phosphonium Cation

In one or more embodiments, one or more of R1, R2, and R3 are antifouling groups comprising one or more sulfonate compounds. As used in this disclosure, sulfonate antifouling agents include any compound comprising the structure depicted in Chemical Structure #5, where R represents a chemical group, which may contain other moieties. R₁₀ may represent branched or unbranched alkanes, alkenes, aryls, or halides.

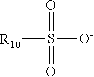

### Chemical Structure #5—Generalized Sulfonate Anion

In one or more embodiments, one or more of R1, R2, and R3 are antifouling groups comprising one or more sulfonium compounds. As used in this disclosure, sulfonium antifouling agents include any compound comprising the sulfonium structure depicted in Chemical Structure #6, where R₁₁, R₁₂, and R₁₃, represents chemical groups which may contain other moieties. R₁₁, R₁₂, and R₁₃ may represent branched or unbranched alkanes, alkenes, aryls, or halides and the R groups may be identical or different from one another.

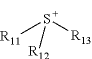

### Chemical Structure #6—Generalized Sulfonium Cation

In one or more embodiments, one or more of R1, R2, and R3 are antifouling groups comprising one or more esters or anhydride compounds. As used in this disclosure, ester antifouling agents include any compound comprising an ester structure and anhydride antifouling agents include any compound comprising an anhydride structure. In some embodiments, the ester or anhydride antifouling group comprises a cyclic moiety. Suitable ester or anhydride antifouling groups which contain a cyclic moiety may include, without limitation, ε-caprolactone, 2-phenylethyl acetate, and polyisobutenyl succinic anhydride. In some embodiments, the ester or anhydride moiety is included in the cyclic moiety. However, in other embodiments, the ester or anhydride moiety is separate from the cyclic moiety. Example cyclic moieties include, without limitation, cyclic alkyls, and aryls, but may include any chemical moiety which includes a ringed structure of atoms. In some embodiments, the ester or anhydride antifouling group may be an ester or anhydride-capped polymer that has a number average molecular weight (Mn) of from 150 grams per mole (g/mol) to 200,000 g/mol (for example, from 150 g/mol to 1,000 g/mol, from 150 g/mol to 2,000 g/mol, from 150 g/mol to 3,000 g/mol, from 150 g/mol to 5,000 g/mol, from 150 g/mol to 10,000 g/mol, from 150 g/mol to 50,000 g/mol, from 150 g/mol to 100,000 g/mol, from 150 g/mol to 150,000 g/mol, from 1,000 g/mol to 200,000) g/mol, from 5,000 g/mol to 200,000 g/mol, from 10,000 g/mol to 200,000 g/mol, from 50,000 g/mol to 200,000 g/mol, or from 100,000 g/mol to 200,000 g/mol).

In one or more embodiments, one or more of R1, R2, and R3 are antifouling groups comprising a polyether compound. As used in this disclosure, polyether antifouling agents include any compound comprising a polyether structure depicted in Chemical Structure #7, where R14 represents chemical groups which may contain other moieties. The polyether antifouling antifouling group may include monomer units comprising carbon chains with one, two, three, four, or even more carbons separating ether moieties. For example, one polyether contemplated in this disclosure includes that depicted in Chemical Structure #7, where j is equal to from 1 to 10 (for example, 1, 2, 3, 4, 5, 6, 7, 8, 9, or 10, or even more, such as j equal to at least 10, at least 25, at least 50, or at least 75, and less than or equal to 100), and k is from 1 to 50,000. R₁₄ in Chemical Structure #7 may represent a hydrogen atom, or an alkyl with or without branches or substitutions. In embodiments, R₁₄ may include at least 5, at least 10, or even more carbon atoms. For example, a suitable polyether antifouling group may be polytetrahydrofuran (where j=4). According to one or more embodiments, the polyether antifouling group may have a number average molecular weight (Mn) of from 150 grams per mole (g/mol) to 200,000 g/mol (for example, from 150 g/mol to 1,000 g/mol, from 150 g/mol to 2,000 g/mol, from 150 g/mol to 3,000 g/mol, from 150 g/mol to 5,000 g/mol, from 150 g/mol to 10,000 g/mol, from 150 g/mol to 50,000 g/mol, from 150 g/mol to 100,000 g/mol, from 150 g/mol to 150,000 g/mol, from 1,000 g/mol to 200,000 g/mol, from 5,000 g/mol to 200,000 g/mol, from 10,000 g/mol to 200,000 g/mol, from 50,000 g/mol to 200,000 g/mol, or from 100,000 g/mol to 200,000 g/mol).

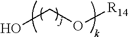

### Chemical Structure #7—Example Polyether Antifouling Group

In one or more embodiments, one or more of R1, R2, and R3 are antifouling groups comprising one or more long-chained amine-capped compounds. As used in this disclosure, long-chained amine-capped antifouling agents include any compound comprising a long-chained amine-capped compound. In one or more embodiments, the long-chained amine-capped antifouling group may have a number average molecular weight (Mn) of from 150 grams per mole (g/mol) to 200,000 g/mol (for example, from 150 g/mol to 1,000 g/mol, from 150 g/mol to 2,000 g/mol, from 150 g/mol to 3,000 g/mol, from 150 g/mol to 5,000 g/mol, from 150 g/mol to 10,000 g/mol, from 150 g/mol to 50,000 g/mol, from 150 g/mol to 100,000 g/mol, from 150 g/mol to 150,000 g/mol, from 1,000 g/mol to 200,000 g/mol, from 5,000 g/mol to 200,000 g/mol, from 10,000 g/mol to 200,000 g/mol, from 50,000 g/mol to 200,000 g/mol, or from 100,000 g/mol to 200,000 g/mol). Suitable long-chained amine-capped antifouling groups include, without limitation, polyisobutene-mono-succinimide and polyisobutene-his-succinimide.

As previously described in this disclosure, one, two, or all three of R1, R2, and R3 may comprise the antifouling groups comprising the structure of Chemical Structure #3, #4, #5, #6, #7, an ester, or an anhydride. In embodiments described in this disclosure, the chemical groups R1, R2, or R3 that do not comprise one of the antifouling groups, if any, are hydrocarbyl groups. For example, R1 may be an antifouling group with the structure depicted in Chemical Structure #3 and R2 and R3 may be hydrocarbyl groups. In another embodiment, R1 and R2 may be antifouling groups with the structure depicted in Chemical Structure #3, and R3 may be a hydrocarbyl group. In another embodiment, R1, R2, and R3 may be antifouling groups with the structure depicted in Chemical Structure #3. When at least two of R1, R2, and R3 are hydrocarbyl groups, they may be identical to one another or may be different hydrocarbyl groups. Also, when two or more of R1, R2, or R3 are antifouling groups, the antifouling groups may be identical or chemically different. However, they will each have the generic structure depicted in Chemical Structure #3, #4, #5, #6, #7, an ester, or an anhydride. R1, R2 and R3 that are hydrocarbyl groups may each have from 1 to 100 carbon atoms, such as, for example, from 1 to 50 carbon atoms. For example, if R1, R2, or R3 are hydrocarbyl groups, they may be straight chained alkanes such as methyl, ethyl, propyl, or butyl groups.

By way of example, if R1 is an antifouling group as depicted in Chemical Structure #3, and R2 and R3 are hydrocarbyl groups, the generalized structure of the antifouling agent can be represented by Chemical Structure #8. It will be appreciated that Chemical Structure #8 may be represented with the formula of (R1)ₓ(R4)_{y}Al—{O[(CH₂)ₙO]ₘR3}_{z}. In various embodiments, the ratio of (x+y)/z may be from 1 to 20, 1 to 10, 1 to 5, 1 to 3 or 2. Further, in various embodiments, the ratio of Al to z may be from 0.01 to 100 as well as the ranges formed from such bounds including 0.05 to 20 and 0.1 to 10.

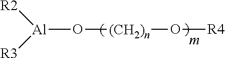

### Chemical Structure #8—Example of Generalized Antifouling Agent

In one or more embodiments, the antifouling agent may comprise an R1 group that is an ethyl group, an R2 group that is an ethyl group, and an R3 that is an antifouling group having the structure —O((CH₂)ₙO)ₘR4 as generally depicted in Chemical Structure #3, where n=2, m=4, and R4 is a dodeyl group. Such an antifouling agent can be written as (CH₃CH₂)₂AlO(CH₂CH₂O)₄(CH₂)₁₁CH₃, and has the chemical structure depicted in Chemical Structure #9, where “Et” represents an ethyl group.

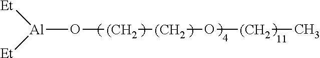

### Chemical Structure #9—Example of Antifouling Agent

In one or more embodiments, the antifouling agent may be present as a dimerized form, referred to herein as an example of a derivative of an antifouling agent. A prepared antifouling agent may be present in both dimerized and non-dimerized (that is, non-bonded) form. For example, in a dimerized state, the antifouling agent may comprise a structure as shown in Chemical Structure #10. Chemical Structure #10 shows the dimerized embodiment of the antifouling agent structure depicted in Chemical Structure #8. In a dimerized embodiment, a bond may form between a central aluminum atom of an antifouling agent molecule and an oxygen atom of a neighboring antifouling agent molecule. It should be understood that while in Chemical Structures #10 the central aluminum atoms are bonded to the oxygen atom in the neighboring antifouling agent that is most near to its central aluminum atom, in other embodiments, this may not be the case, and the a central aluminum atom may bond with an oxygen atom of a neighboring antifouling agent which is not most near to its central aluminum atom.

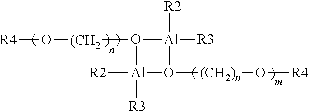

### Chemical Structure #10—Example of Dimerized Antifouling Agent

In one or more embodiments, the antifouling agent may be present in different isomer states, one such example depicted in Chemical Structure #11. An isomer is an example of a derivative structure of an antifouling agent. For example, and as depicted in Chemical Structure #11, the central aluminum atom of an antifouling agent may be bonded to two oxygens atoms of a single antifouling group. It should be understood that while Chemical Structure #11 depicts an isomer where the two oxygen atoms most near to the central aluminum atom are bonded with the central aluminum atom, in other embodiments other isomers may form, such as an isomer formed when the central aluminum atom forms a bond with an oxygen atom which is not as close as another oxygen atom to the central aluminum atom in the antifouling agent molecule. For example, while Chemical Structure #11 shows a ring structure with 2 oxygen atoms and n carbon atoms, larger ring structures may form in other isomers, such as rings having three or more oxygen atoms. It should be understood that isomers of the antifouling agents described, such as that shown in Chemical Structure #11, are considered antifouling agents and fit into the base structure depicted in Chemical Structure #2. For instance, the existence of two oxygen atoms bonded to the central aluminum, where both oxygen atoms are part of an antifouling group, is considered to conform to the base structure depicted in Chemical Structure #2.

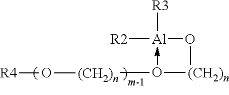

### Chemical Structure #11—Example of Isomer of Antifouling Agent

In one or more embodiments, the antifouling catalyst systems may comprise more than one molecular species of antifouling agent. For example, the antifouling catalyst system may comprise antifouling agents with a varying number of antifouling groups. For example, some antifouling agent molecules may comprise none, one, two or three antifouling groups, while others comprise a different number of antifouling groups. The mixture of these antifouling agent species may form a bulk antifouling agent which can be characterized by its bulk molar ratio of hydrocarbyl groups to antifouling groups which are attached to the central aluminum atoms, respectively. For example, if half of the antifouling agent has one antifouling group and two hydrocarbyl groups, and, the other half of the antifouling agent has two antifouling groups and one hydrocarbyl group, then the bulk molar ratio of hydrocarbyl groups to antifouling groups would be 1:1 because there is a bulk equal amount of hydrocarbyl groups to antifouling groups. In one or more embodiments, the bulk molar ratio of hydrocarbyl groups to antifouling groups may be from be from 1:1 to 20:1. Non-limiting examples of bulk molar ratios of hydrocarbyl groups to antifouling groups include from 1:1 to 2:1, from 1:1 to 3:1, from 1:1 to 4:1, from 1:1 to 5:1, from 1:1 to 10:1, from 1:1 to 15:1, from 2:1 to 20:1, from 3:1 to 20:1, from 4:1 to 20:1, from 5:1 to 20:1, from 10:1 to 20:1, or from 15:1 to 20:1. According to one or more embodiments, the bulk molar ratio of hydrocarbyl groups to antifouling groups is from 1.5 to 2.5, from 1.8 to 2.2, or 2.

In one or more embodiments, the antifouling catalyst system may comprise one or more nitrogen containing compounds. The one or more nitrogen containing compounds may include one or both of a primary or a secondary amine. Non-limiting examples of the primary amines may include ethylamine, isopropylamine, cyclohexylamine, benzylamine, aniline, and naphthylamine. Non-limiting examples of the Secondary amines may include diethylamine, diisopropylamine, dicyclohexylamine, dibenzylamine, bis(trimethylsilyl) amine, morpholine, imidazole, indoline, indole, pyrrole, 2,5-dimethyl-pyrrole, 3,4-dimethyl-pyrrole, 3,4-dichloro-pyrrole, 2,3,4,5-tetrachloro-pyrrole, 2-acyl pyrrole, pyrazole, and pyrrolidine.

In one or more embodiments, the antifouling catalyst system may comprise one or more halogen containing compounds. The halogen-containing compounds are the compounds containing an element selected from those belonging to the 3-, 4-, 5-, 6-, 13-, 14- and 15-Groups of the Periodic Table. The one or more halogen containing compounds may include CHX₃, CH₂X₂, C₂X₆, Et₂Al_{X}, Et₂Al₂X₃, TiX₄, GeX₄, SnX₄, or combinations thereof, where X is F, or Br and Et is an ethyl group. Further, specific and non-limiting examples of the afore-mentioned halogen containing compound include scandium chloride, yttrium chloride, lanthanum chloride, titanium tetrachloride, zirconium tetrachloride, hafnium tetrachloride, molybdenum chloride, manganese chloride, boron trichloride, aluminum chloride, diethyl aluminum chloride, ethyl aluminum sesquichloride, gallium chloride, carbon tetrachloride, chloroform, methylene chloride, dichloroethane, hexachlorobenzene, 1,3,5-trichlorobenzene, trityl chloride, silane tetrachloride, trimethyl-chlorosilane, germanium tetrachloride, tin tetrachloride, tributyl-tin chloride, phosphorus trichloride, antimony trichloride, trityl-hexachloroantimonate, antimony pentachloride, bismuth trichloride, boron tribromide, aluminum tribromide, carbon tetrabromide, bromoform, bromobenzene, iodomethane, silicon tetrabromide, hexafluoro benzene, and aluminum fluoride. In one or more embodiments, the halogen containing compounds may be used in the form of a mixture of 2 or more different compounds.

The antifouling catalyst systems may comprise at least one or more chromium compounds, one or more aluminum compounds, and one or more antifouling agents. In one or more embodiments, the molar ratio of total chromium compound to total aluminum compound may be from 1:1 to 1:5,000. In various embodiments, the molar ratio of total chromium compound to total aluminum compound may be from 1:1 to 1:2,500, from 1:1 to 1:1,000, from 1:1 to 1:500, from 1:1 to 1:250, 1:5 to 1:2,500, from 1:10 to 1:5,000, from 1:10 to 1:1,000, from 1:10 to 1:500, or from 1:10 to 1:100.

In one or more embodiments, the molar ratio of total chromium compounds to total antifouling agent may be from 1:100 to 1:0.001. In various embodiments, the molar ratio of total chromium compounds to total antifouling agent may be from 1:10 to 1:0.01, from 1:10 to 1:0.05, from 1:10 to 1:0.3, from 1:10 to 1:0.5, from 1:10 to 1:0.7, from 1:10 to 1:1, from 1:5 to 1:0.01, from 1:3 to 1:0.01, from 1:2 to 1:0.1, or from 1:1 to 1:0.1.

In one or more embodiments, the molar ratio of total chromium compounds to total nitrogen containing compounds may be from 1:10 to 1:0.01 In various embodiments, the molar ratio of total chromium compounds to total nitrogen containing compounds may be from 1:8 to 1:0.1, from 1:6 to 1:0.1, from 1:5 to 1:0.1, or from 1:2 to 1:0.1, from 1:0.5 to 1:0, from 1:0.3 to 1:0, or from 1:0.1 to 1:0.1.

It should be understood that the molar ratios of components of the antifouling catalyst systems described previously are representative of the total amount of each component of the antifouling catalyst system relative to the total amount of chromium compound, where the “total” amount refers to the molar amount of all species of the antifouling catalyst system which may be considered as a particular component type (that is, chromium compound, aluminum compound, nitrogen containing compound, halogen containing compound, or antifouling agent). The total amount of a component may include two or more chemical species which are chromium compounds, aluminum compounds, nitrogen containing compounds, halogen containing compounds, or antifouling agents, respectively. It should also be understood that, as used in this disclosure, the total amount of aluminum compound does not include molecules that are considered as antifouling agents. Therefore, any species that is considered an antifouling agent as described in this disclosure does not contribute towards the total amount of aluminum compound even though the antifouling agent includes an aluminum central atom and may otherwise be considered as an aluminum-containing compound.

In one or more embodiments, the antifouling agent does not dealkylate, or is resistant to dealkylation as compared with other catalyst systems. Ethane releasing reactions may be undesirable, as they can contaminate a feed stream. In one or more embodiments, the antifouling agent does not deactivate the catalytic centers of one or more of the chromium compound or the aluminum compound.

According to another embodiment of the present disclosure, 1-hexene may be produced. According to the method for 1-hexene production, ethylene may be contacted with the antifouling catalyst system described previously to oligomerize the ethylene to form 1-hexene. In one or more embodiments, the ethylene and antifouling catalyst system are supplied to a reactor and mixed. The reaction may be performed as a batch reaction or as a continuous process reaction, such as a continuous stir tank reactor process. According to embodiments, the pressure of the reactor may be from 2 bar to 100 bar and the reactor temperature may be from 30 degrees Celsius (° C.) to 120° C. However, process conditions outside of these ranges are contemplated, especially in view of the specific design of the reactor system and concentrations of the reactants and catalysts.

In one or more embodiments, the introduction of the antifouling agent into a catalyst system may suppress polymer formation while not greatly reducing catalytic activity of 1-hexene formation, in one embodiment, polymer formation (fouling) may be reduced by at least 5%, 10%, 20%, 30%, 40%, 50%, 60%, 70%, 80%, 90%, or even 95% by the inclusion of an antifouling agent. In one embodiment, 1-hexene production may be increased, stay the same, or may decrease by less than or equal to 50%, 40%, 30%, 20%, 10% or even 5% by the inclusion of an antifouling agent. In some embodiments, antifouling agents may both reduce the polymer formation (such as by at least 5%, 10%, 20%, 30%, 40%, 50%, 60%, 70%, 80%, 90% or even 95%) and increase, not effect, or decrease 1-hexene production rate by less than or equal to 50%, 40%, 30%, 20%, 10% or even 5%. Reduction in polymer formation rates and catalytic activity on a percentage basis are based on catalyst systems which include one or more antifouling agents described as compared with catalyst systems which are void of an antifouling agent.

### EXAMPLES

The various embodiments of antifouling catalyst systems will be further clarified by the following examples. The examples are illustrative in nature, and should not be understood to limit the subject matter of the present disclosure.

To form an antifouling agent having the chemical structure (CH₃CH₂)₂AlO(CH₂CH₂O)₄C₁₂H₂₅ (shown as Chemical Structure #9), 10 milliliters (mL) of triethyl aluminum (1 molar (M) in hexane) was reacted with 10 mL of polyethylene glycol dodecyl ether (1 M in hexane). Specifically, polyethylene glycol dodecyl ether was first dried with anhydrous grade sodium sulfate to remove any residual moisture. The dried polyethylene dodecyl ether was then added drop-wise to the triethyl aluminum in a 30 mL flask. The reaction mixture was subsequently stirred for 15 minutes. The reaction was carried out in an inert atmosphere inside a glove box. Light color change and release of ethane gas were observed during the reaction. Other antifouling agents were produced though similar techniques which incorporated reactants suitable to achieve antifouling agents having varying values of n, in, and R4 as described in this disclosure with reference to antifouling groups.

It will be appreciated that the antifouling agent is indicated as having the chemical structure as shown in Chemical Structure #9, yet the isomer and dimer of the same may also be present as illustrated in Chemical Structure #12 and Chemical Structure #13 respectively.

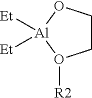

### Chemical Structure #12—Example of Isomer of Antifouling Agent

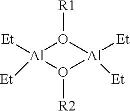

### Chemical Structure #13—Example of Dimerized Antifouling Agent

To evaluate the anti-fouling effects of the antifouling catalyst systems described, ethylene oligomerization reactions were carried out and evaluated. Multiple sample antifouling catalyst systems were formulated. Detailed structure of the antifouling agents used, as well as ratios of are listed in Table 1. For the experiments, catalyst mixtures were used that contained a complex of chromium and 2-ethyl hexanoate (denoted as “Cr” in Table 1) on molar basis of “Cr”, 2,5 dimethyl pyrrole (denoted as “DMP” in Table 1) (nitrogen containing compound), triethyl aluminum (denoted as “TEAL” in Table 1), diethyl aluminum chloride (denoted as “DEAC” in Table 1) (halogen containing compound), and antifouling agents (denoted as “AFA” in Table 1). The antifouling agents utilized are described previously in this disclosure and are depicted in Chemical Structure #9. In addition to the catalyst mixtures containing antifouling agent, a comparative example which did not contain antifouling agent was tested and compared with the catalyst systems which included antifouling agents.

The components of the sample catalyst systems were prepared and transferred into metal charging cylinders in a glove box. The complex of chromium and 2-ethyl hexanoate was premixed with the 2,5 dimethyl pyrrole to form a chromium pyrolide complex (Mixture A). Similarly, the triethyl aluminum (TEAL) was premised with the diethyl aluminum chloride (DEAC) to form a precursor mixture (Mixture B). Finally, the antifouling agent was premixed with n-hexane (0.001M to 1.0 M) utilized as an anhydrous solvent. Cr concentration used in the examples was approximately 16 μM and the AFA concentration was maintained between 0.8 μM and 4.8 μM.

The oligomerization reactions were conducted in an autoclave batch reactor unit (250 mL volume). In a typical reaction run, the reactor vessel was vacuum purged with ultrapure nitrogen to remove oxygen and moisture. Then, the batch reactor was filled with the mixture of the anhydrous n-hexane and antifouling agent as well as Mixture B and kept at 35-120° C., desireably 40-100° C., and more desirably at 45-80° C. Then, the chromium pyrolide complex (Mixture A) was introduced into the reactor. It will be appreciated that Mixture A, Mixture B, and the mixture of the anhydrous n-hexane and antifouling agent may all be pre-mixed together and provided to the reactor as a single solution. Following introduction of the components of the catalyst system, the reactor was pressurized to 4 mega-Pascals (MPa) (40 bar) with ethylene and the temperature of the reactor was set to 120° C. with a stirring rate of 1500 rpm. The trimerization reaction was terminated by injecting 2 mL of methanol after 120 minutes. It will be appreciated that methanol may also be used to terminate the reaction. The reactor was subsequently depressurized. The remaining solid polymer was filtered, dried overnight in an oven at 110° C. and weighed.

Table 1 shows the trimerization activity and polymer formation for reactions which utilized each of the sample catalyst systems. As is evident by the reaction data of Table 1, the addition of the antifouling additive greatly reduced polymer formation and increased 1-hexene formation.

TABLE 1

| | | | Activity (moles of | Polymer | 1-hexene |
|---|---|---|---|---|---|
| | Molar Ratio of | Molar Ratio of | 1-hexene per mole | (wt. %) on | selectivity |
| | AFA:Cr | Cr:DMP:DEAC:TEAL | of chromium per hour) | dry basis | (mol. %) |
| Inventive | 0.01 | 1:3:12:16.5 | 6966.00 | 0.60 | 96.29 |
| Example 1 | | | | | |
| Inventive | 0.017 | 1:3:12:16.5 | 7189.18 | 0.45 | 96.30 |
| Example 2 | | | | | |
| Inventive | 0.025 | 1:3:12:16.5 | 7185.97 | 0.30 | 96.28 |
| Example 3 | | | | | |
| Inventive | 0.035 | 1:3:12:16.5 | 6120.30 | 0.27 | 96.27 |
| Example 4 | | | | | |
| Inventive | 0.05 | 1:3:12:16.5 | 5669.61 | 0.22 | 96.22 |
| Example 5 | | | | | |
| Comparative | 0 | 1:3:12:16.5 | 6885.26 | 1.29 | 94.71 |
| Example 6 | | | | | |

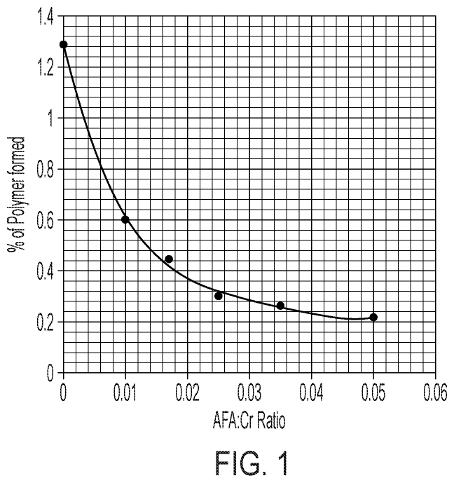

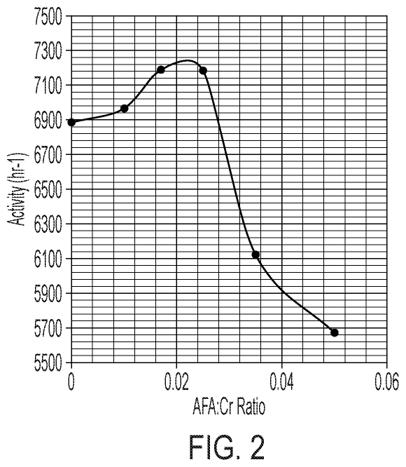

The effect of the AFA:Cr ratio on polymer formation is graphically illustrated in FIG. 1. Similarly, the effect of the AFA:Cr ratio on the trimerization activity rate is graphically illustrated in FIG. 2. Finally, the effect of the AFA:Cr ratio on 1-hexene selectivity is graphically illustrated in HG 3.

It should now be understood the various aspects of the catalyst system that reduces polymeric fouling in the production of 1-hexene and associated method of producing 1-hexene are described and such aspects may be utilized in conjunction with various other aspects.

According to a first aspect, a catalyst system that reduces polymeric fouling in the production of 1-hexene includes at least one chromium compound, at least one aluminum compound, and at least one antifouling agent or a derivative thereof. The antifouling agent comprises the structure of Chemical Structure #2 where one or more of the chemical groups R1, R2, and R3 are antifouling groups. The antifouling groups comprising a phosphonium or phosphonium salt, a sulfonate or sulfonate salt, a sulfonium or sulfonium salt, an ester, an anhydride, a polyether, a long-chained amine-capped compound, or the structure —O((CH₂)ₙO)ₘR4, where n is an integer from 1 to 20; m is an integer from 1 to 100, and R4 is a hydrocarbyl group. Further the chemical groups R1, R2, or R3 that do not comprise the antifouling group, if any, are hydrocarbyl groups.

A second aspect includes the catalyst system of the first aspect in which n is from 1 to 5.

A third aspect includes the catalyst system of the first or second aspects in which m is from 1 to 20.

A fourth aspect includes the catalyst system of any of the first through third aspects in which R4 has from 1 to 100 carbon atoms.

A fifth aspect includes the catalyst system of any of the first through fourth aspects in which at least one of the chromium compounds is a chromium complex chosen from alkoxy salts of chromium, carboxyl salts of chromium, B-diketonate salts of chromium, salts of chromium and an anion derived from B-keto-esters, or chromium halides.

A sixth aspect includes the catalyst system of the fifth aspect in which the chromium complex is chosen from chromium (IV)-tert-butoxide, chromium (III)-acetylacetonate, chromium (III)-trifluoro acetylacetonate, chromium (III)-hexafluoro acetylacetonate, chromium (III) (2,2,6,6-tetramethyl-3,5-heptane-dionate), Cr(PhCOCHCOPh) wherein Ph represents a phenyl group, chromium (II) acetate, chromium (III) acetate, chromium (III)-2-ethyl hexanoate, chromium (III) benzoate, chromium (III) naphthenate, Cr(CH—COCHCOOCH), chromium (II) chloride, chromium(III) chloride, chromium (II) bromide, chromium (III)bromide, chromium(II) iodide, chromium(III) iodide, chromium (II) fluoride, and chromium (III) fluoride.

A seventh aspect includes the catalyst system of any of the first through sixth aspects in which at least one of the aluminum compounds has the structure AlR′₃ or AlR′₂H, where R′ is a branched or straight chain alkyl radical comprising from 2 to 8 carbon atoms.

An eighth aspect includes the catalyst system of any of the first through seventh aspects in which at least one of the aluminum compounds is chosen from triethylaluminum, tripropylaluminum, tri-iso-butylaluminum, trihexylaluminum, or an aluminoxane.

A ninth aspect includes the catalyst system of any of the first through eighth aspects in which the system further comprises a nitrogen containing compound, the nitrogen containing compound being a primary or secondary amine.

A tenth aspect includes the catalyst system of the ninth aspect in which the nitrogen containing compound is selected from ethylamine, isopropylamine, cyclohexylamine, benzylamine, aniline, naphthylamine, diethylamine, diisopropylamine, dicyclohexylamine, dibenzylamine, bis(trimethylsilyl) amine, morpholine, imidazole, indoline, indole, pyrrole, 2,5-dimethyl-pyrrole, 3,4-dimethyl-pyrrole, 3,4-dichloro-pyrrole, 2,3,4,5-tetrachloro-pyrrole, 2-acyl pyrrole, pyrazole, and pyrrolidine.

An eleventh aspect includes the catalyst system of any of the first through tenth aspects in which the system further comprises a halogen containing compound.

A twelfth aspect includes the catalyst system of the eleventh in which the halogen containing compound comprises CHX₃, CH₂X₂, C₂X₆, Et₂Al_{X}, TiX₄, GeX₄, SnX₄, or combinations thereof, where X is F, Cl, or Br and Et is an ethyl group.

A thirteenth aspect includes the catalyst system of any of the first through twelfth aspects in which a molar ratio of total chromium compound to total aluminum compound is from 1:1 to 1:5000.

A fourteenth aspect includes the catalyst system of any of the first through thirteenth aspects in which a molar ratio of total chromium compound to total antifouling agent is from 1:10 to 1:0.01.

A fifteenth aspect includes the catalyst system of any of the first through fourteenth aspects in which a molar ratio of total chromium compound to total nitrogen containing compound is from 1:10 to 1:0.

According to a sixteenth aspect, a method of producing 1-hexene includes contacting ethylene with a catalyst system according to any of the first through fifteenth aspects to oligomerize the ethylene to form 1-hexene\It should be apparent to those skilled in the art that various modifications and variations can be made to the described embodiments without departing from the spirit and scope of the claimed subject matter. Thus, it is intended that the specification cover the modifications and variations of the various described embodiments provided such modifications and variations come within the scope of the appended claims and their equivalents.

The singular forms “a”, “an” and “the” include plural referents, unless the context clearly dictates otherwise.

Throughout this disclosure ranges are provided. It is envisioned that each discrete value encompassed by the ranges are also included. Additionally, the ranges which may be formed by each discrete value encompassed by the explicitly disclosed ranges are equally envisioned. For brevity, the same is not explicitly indicated subsequent to each disclosed range and the present general indication is provided.

As used in this disclosure and in the appended claims, the words “comprise,” “has,” and “include” and all grammatical variations thereof are each intended to have an open, non-limiting meaning that does not exclude additional elements or steps.

What is claimed is:

1. A catalyst system that reduces polymeric fouling in the production of 1-hexene, the catalyst system comprising:
at least one chromium compound;
at least one aluminum compound; and
at least one antifouling agent or a derivative thereof, where the antifouling agent comprises the structure: 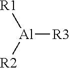
where one or more of the chemical groups R1, R2, and R3 are antifouling groups comprising the structure —O((CH₂)ₙO)ₘR4, a phosphonium or phosphonium salt, a sulfonate or sulfonate salt, a sulfonium or sulfonium salt, an ester, an anhydride, a polyether, or a long-chained amine-capped compound, where:
n is an integer from 1 to 20;
m is an integer from 1 to 100; and
R4 is a hydrocarbyl group; and
where the chemical groups R1, R2, or R3 that do not comprise the antifouling group, if any, are hydrocarbyl groups.

2. The catalyst system of claim 1, where n is from 1 to 5.

3. The catalyst system of claim 1, where m is from 1 to 20.

4. The catalyst system of claim 1, where R4 has from 1 to 100 carbon atoms.

5. The catalyst system of claim 1, where at least one of the chromium compounds is a chromium complex chosen from alkoxy salts of chromium, carboxyl salts of chromium, B-diketonate salts of chromium, salts of chromium and an anion derived from B-keto-esters, or chromium halides.

6. The catalyst system of claim 5, where the chromium complex is chosen from chromium (IV)-tert-butoxide, chromium (III)-acetylacetonate, chromium (III)-trifluoro acetylacetonate, chromium (III)-hexafluoro acetylacetonate, chromium (III) (2,2,6,6-tetramethyl-3,5-heptane-dionate), Cr(PhCOCHCOPh) wherein Ph represents a phenyl group, chromium (II) acetate, chromium (III) acetate, chromium (III)-2-ethyl hexanoate, chromium (III) benzoate, chromium (III) naphthenate, Cr(CH—COCHCOOCH), chromium (II) chloride, chromium(III) chloride, chromium (II) bromide, chromium (III)bromide, chromium(II) iodide, chromium(III) iodide, chromium (II) fluoride, and chromium (III) fluoride.

7. The catalyst system of claim 1, where at least one of the aluminum compounds has the structure AlR′₃ or AlR′₂H, where R′ is a branched or straight chain alkyl radical comprising from 2 to 8 carbon atoms.

8. The catalyst system of claim 1, where at least one of the aluminum compounds is chosen from triethylaluminum, tripropylaluminum, tri-iso-butylaluminum, trihexylaluminum, or an aluminoxane.

9. The catalyst system of claim 1, further comprising a nitrogen containing compound, the nitrogen containing compound being a primary or secondary amine.

10. The catalyst system of claim 9, where the nitrogen containing compound is selected from ethylamine, isopropylamine, cyclohexylamine, benzylamine, aniline, naphthylamine, diethylamine, diisopropylamine, dicyclohexylamine, dibenzylamine, bis(trimethylsilyl) amine, morpholine, imidazole, indoline, indole, pyrrole, 2,5-dimethyl-pyrrole, 3,4-dimethyl-pyrrole, 3,4-dichloro-pyrrole, 2,3,4,5-tetrachloro-pyrrole, 2-acyl pyrrole, pyrazole, and pyrrolidine.

11. The catalyst system of claim 1, further comprising a halogen containing compound.

12. The catalyst system of claim 11, where the halogen containing compound comprises CHX₃, CH₂X₂, C₂X₆, Et₂Al_{X}, Et₂Al₂X₃, TiX₄, GeX₄, SnX₄, or combinations thereof, where X is F, Cl, or Br and Et is an ethyl group.

13. The catalyst system of claim 1, where a molar ratio of total chromium compound to total aluminum compound is from 1:1 to 1:5000.

14. The catalyst system of claim 1, where a molar ratio of total chromium compound to total antifouling agent is from 1:10 to 1:0.01.

15. The catalyst system of claim 1, where a molar ratio of total chromium compound to total nitrogen containing compound is from 1:10 to 1:0.

16. A method of producing 1-hexene comprising:
contacting ethylene with a catalyst system to oligomerize the ethylene to form 1-hexene, where the catalyst system comprises:
at least one chromium compound;
at least one aluminum compound; and
at least one antifouling agent or derivative thereof, where the antifouling agent comprises the structure: 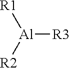
where one or more of the chemical groups R1, R2, and R3 are antifouling groups comprising the structure —O((CH₂)ₙO)ₘR4, a phosphonium or phosphonium salt, a sulfonate or sulfonate salt, a sulfonium or sulfonium salt, an ester, an anhydride, a polyether, or a long-chained amine-capped compound, where:
n is an integer from 1 to 20;
m is an integer from 1 to 100; and
R4 is a hydrocarbyl group;
where the chemical groups R1, R2, or R3 that do not comprise the antifouling group, if any, are hydrocarbyl groups.

17. The catalyst system of claim 16, where at least one of the chromium compounds is a chromium complex chosen from alkoxy salts of chromium, carboxyl salts of chromium, B-diketonate salts of chromium, salts of chromium and an anion derived from B-keto-esters, or chromium halides.

18. The catalyst system of claim 16, where at least one of the aluminum compounds has the structure AlR′₃ or AlR′₂H, where R′ is a branched or straight chain alkyl radical comprising from 2 to 8 carbon atoms.

19. The catalyst system of claim 16, further comprising a nitrogen containing compound, the nitrogen containing compound being a primary or secondary amine.

20. The catalyst system of claim 16, further comprising a halogen containing compound.
